(19) United States
(12) Patent Application Publication
(10) Pub. No.: US 2026/0000673 A1
(43) Pub. Date: Jan. 1, 2026

(54) BRAIN HEALTH SUPPLEMENT

(71) Applicant: Matthias Magoola, Kampala (UG)

(72) Inventor: Matthias Magoola, Kampala (UG)

(21) Appl. No.: 18/760,014

(22) Filed: Jul. 1, 2024

(51) Int. Cl. A61K 31/525; A61K 9/20; A61K 31/4188; A61K 45/06

(52) U.S. Cl. CPC A61K 31/525; A61K 9/2018; A61K 9/2027; A61K 9/2031; A61K 9/2095; A61K 31/4188; A61K 45/06

(57) ABSTRACT

A solid dosage form for daily dosing containing biotin and riboflavin to prevent Parkinson's disease.

### BACKGROUND OF THE INVENTION

Parkinson's disease (PD) shows significant reductions insignificantly reduced biotin biosynthesis. There is a correlation between decreased biosynthetic genes for riboflavin and biotin and lower fecal concentrations of SCFAs and polyamines that can lead to a thinner intestinal mucus layer, facilitating the formation of abnormal α-synuclein fibrils in the intestinal neural plexus and contributing to neuroinflammation in PD. Supplementation with riboflavin and biotin can be beneficial for some PD patients and, when used as a preventive measure, reduce the incidence of Parkinson's disease.

### DETAIL DESCRIPTION

### Bill of Materials

| Scale (mg/ | | | Quantity/1000 |
|---|---|---|---|
| tablet) | Item | Material Name | Tablets (g) |
| 95.00 | 1 | Thiamine mononitrate | 95.00 |
| 20.00 | 2 | Riboflavin (B7) | 20.00 |
| 100.00 | 3 | Nicotinamide | 100.00 |
| 50.00 | 4 | Calcium D-pantothenate | 50.00 |
| 2.00 | 5 | Folic acid | 2.00 |
| 0.20 | 6 | Biotin | 0.20 |
| 0.005 | 7 | Cyanocobalamin (gelatin coated, 1%) | 0.50 |
| 50.00 | 8 | Carnitine hydrochloride | 50.00 |
| 100.00 | 9 | Inositol | 100.00 |
| 2.00 | 10 | Adenosine phosphate | 2.00 |
| 15.70 | 11 | Kollidon QR 30 | 15.70 |
| 70.00 | 12 | Isopropanol | 70.00 |
| 26.00 | 13 | Kollidon QR CL | 26.00 |
| 122.00 | 14 | Lactose monohydrate | 122.00 |
| 14.00 | 15 | PEG-6000, powder | 14.00 |

Manufacturing Directions: Granulate mixture of items 1 to 10 with a solution of items 11 and 12; Dry, pass through a 0.8-mm sieve, mix with items 13 and 15, and press with low compressive force; Compress into 708-mg tablets, using 13-mm biplanar punches.

1. An oral solid dosage form to prevent or treat Parkinson's disease contains an effective dose of riboflavin, biotin, and inactive ingredients to formulate the dosage form and, optionally, other nutrients.

2. The oral solid dosage form to prevent or treat Parkinson's disease of claim 1, wherein the dosage form optionally contains nicotinamide, calcium D-pantothenate, folic acid, cyanocobalamin, carnitine, inositol, thiamine, and adenosine phosphate.

3. The oral solid dosage form is used to prevent or treat Parkinson's disease of claim 1, wherein the dosage form contains inactive ingredients consisting of Kollidons, Lactose, isopropyl alcohol, and polyethylene glycol.

4. The oral solid dosage form is used to prevent or treat Parkinson's disease of claim 1, wherein the dosage form is a tablet, a capsule, or a powder.

5. The oral solid dosage form to prevent or treat Parkinson's disease of claim 4, wherein a single unit weighs 703 mg as a tablet compressed with 13-mm biplanar punches.

6. The oral solid dosage form to prevent or treat Parkinson's disease of claim 1, wherein the single unit contains 20 mg of riboflavin and 0.2 mg of biotin.

7. The oral solid dosage form to prevent or treat Parkinson's disease of claim 2, wherein the single unit additionally contains 95 mg of thiamine mononitrate, 100 mg of nicotinamide, 50 mg of calcium D-pantothenate, 2 mg of folic acid, 0.005 mg of cyanocobalamin, 50 mg of carnitine hydrochloride, and 2 mg of adenosine phosphate.

8. The oral solid dosage form to prevent or treat Parkinson's disease of claim 1, wherein a single unit is dosed daily to adult humans.
